(19) United States
(12) Patent Application Publication
(10) Pub. No.: US 2007/0088177 A1
(43) Pub. Date: Apr. 19, 2007

(54) Process for preparing dimethyl cyanimidocarbonate

(76) Inventors: Holger Weintritt, Langenfeld (DE); Michael Bauer, Pulheim (DE); Armin Heyn, Bergisch Gladbach (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/556,813

(22) PCT Filed: Apr. 30, 2004
(86) PCT No.: PCT/EP04/04594

§ 371 Date: Nov. 27, 2006

(30) Foreign Application Priority Data
May 13, 2003 (DE) 103 21 269.8

(51) Int. Cl. C07C 261/04

(52) U.S. Cl. 564/106

(57) ABSTRACT

The invention relates to a novel process for preparing dimethyl cyanimidocarbonate (DCC, 3,3-dimethoxy-2-azaprop-2-enenitrile) from sodium cyanide, methanol, chlorine gas and cyanamide.

The invention relates to a novel process for preparing dimethyl cyanimidocarbonate (DCC, 3,3-dimethoxy-2-azaprop-2-enenitrile).

It is already known that diethyl cyanimidocarbonate can be prepared by reacting isolated diethyl imidocarbonate with cyanamide under anhydrous conditions (Chem. Ber. 1967, 100, 2604). However, the yield of this process is unsatisfactory, requiring an additional work-up step.

Furthermore, it is known that dimethyl cyanimidocarbonate can be prepared by reacting an appropriate imidocarbonate with cyanamide in a two-phase system comprising water and a water-immiscible organic solvent, for example toluene (EP-A 0 014 064).

Moreover, it is known that dimethyl cyanimidocarbonate can be prepared by initially reacting sodium cyanide under alkaline conditions with methanol, then introducing chlorine and, after neutralization of the reaction mixture and addition of cyanamide, recovering the substituted N-cyanimidocarbonate formed after addition of methylene chloride from the organic phase (DE-A 32 25 249).

Furthermore, it is known that dimethyl cyanimidocarbonate is obtained by initially preparing the appropriate imidocarbonate from methanol and cyanogen chloride, followed by addition of the imidocarbonate and an acid to an initial cyanamide solution charge (EP-B 0 523 619).

The processes described have the disadvantages that in their practice either harmful by-products are formed or it is necessary to use relatively large amounts of organic solvents (for example toluene) or reagents difficult to handle on an industrial scale (for example cyanogen chloride) are employed.

Accordingly, it was an object of the present invention to develop a process which is easy to realize on an industrial scale and which affords DCC in good yields and high purity.

It has now been found that dimethyl cyanimidocarbonate (DCC, 3,3-dimethoxy-2-azaprop-2-enenitrile) of the formula (I) 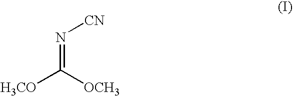
is obtained when,
in a first step sodium cyanide is reacted in aqueous sodium hydroxide solution with methanol and chlorine gas and,
in a second step, the resulting dimethyl imidocarbonate (dimethoxymethanimine) of the formula (II) 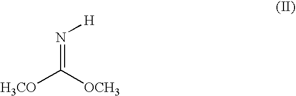
   is neutralized and,
in a third step, the resulting neutralized dimethyl imidocarbonate of the formula (II) is introduced into an aqueous solution of cyanamide (aminomethanenitrile), the pH being kept neutral by simultaneous addition of an acid.

For work-up, in a fourth step, by-products with oxidative action are reduced in the presence of an extractant,
in a fifth step, non-product-containing solid particles are removed by clarification and,
in a sixth step, the DCC of the formula (I) is concentrated by extraction and distillation.

Surprisingly, the process according to the invention affords DCC in a simple manner in high purity. It is particularly surprising that the neutralized dimethyl imidocarbonate is stable for sufficiently long under the chosen reaction conditions for it to be used for the next reaction without loss of yield.

In addition, the reaction according to the invention has the advantage of being more environmentally friendly and safer since there is no need to use large amounts of operating fluids or reactants which may be hazardous to health.

The starting materials sodium cyanide, aqueous sodium hydroxide solution, methanol, chlorine gas, cyanamide and sodium hydrogensulphite are known chemicals.

To neutralize the dimethyl imidocarbonate in the second step and to keep the pH in the third step neutral, an acid suitable for these purposes, preferably hydrochloric acid, is employed.

When carrying out the process according to the invention, the reaction temperatures can be varied within a relatively wide range. The first step is generally carried out at temperatures between −50° C. and 0° C. preferably between −25° C. and 0°C. particularly preferably at −5° C. The second step is generally carried out at temperatures between −20° C. and 0° C. preferably between −10° C. and 0° C. particularly preferably at −5° C. The third step is generally carried out at temperatures between −20° C. and +30° C. preferably between −5° C. and +20° C.

When carrying out the first step of the process according to the invention, in general from 0.8 to 1.5 mol, preferably from 0.9 to 1.3 mol, particularly preferably from 1.0 to 1.2 mol, of sodium hydroxide and generally from 2 to 10 mol, preferably from 2 to 5 mol, particularly preferably from 3 to 4 mol, of methanol and generally from 0.8 to 0.97 mol, preferably from 0.85 to 0.95 mol, particularly preferably from 0.90 to 0.95 mol, of chlorine are employed per mole of sodium cyanide.

Chlorine is preferably employed in slightly substoichiometric amounts to keep the formation of unwanted by-products at a minimum. Surprisingly, it has been found that higher and more consistent yields are obtained when the chlorine is introduced above the reaction mixture than when the chlorine is introduced into the reaction mixture.

Here, “introduction above the reaction mixture” for the purpose of the invention is to be understood as meaning that the chlorine is introduced into the gas space above the liquid reaction mixture, whereas during “introduction into the reaction mixture” the end of the gas inlet tube is below the surface of the liquid. Accordingly, during introduction above the reaction mixture, concentration peaks in the gas inlet apparatus are avoided. The gas (here: chlorine) is taken up via the surface of the liquid reaction mixture.

The reaction time in the first step is not critical and is from a few minutes to several hours. Depending on the size of the batch and the heat dissipation, the time for introducing the chlorine gas above the reaction mixture is between 1 h and 20 h, generally between 5 h and 10 h.

When carrying out the second step of the process according to the invention, in general from 0.5 to 1.5 mol, preferably from 0.6 to 0.9 mol, of hydrochloric acid are employed per mole of sodium cyanide. However, it is also possible to choose other ratios.

Neutralization in the practice of the second step of the process according to the invention is complete when the reaction mixture has reached a pH in the range of from pH 6.5 to pH 7.5, preferably from pH6.8to pH7.2.

The neutralization of the dimethyl imidocarbonate in the second step is preferably carried out continuously, with residence times of at most 30 min.

Continuous operation is advantageous since in this manner the neutralized dimethyl imidocarbonate remains sufficiently stable, so that it can be used without loss of yield for the next reaction. In principle, this would also be possible with batch-wise operation; however, here the expected yields are lower than in the case of continuous operation.

The continuous neutralization is carried out in a loop reactor having a suitable circulation ratio between the circulated volume stream and the volume stream removed from the loop, so that the high heat of neutralization at the preferred residence times in the loop can be dissipated. Here, the control systems for continuously metering hydrochloric acid into the loop are adjusted such that the desired pH range can be maintained for the entire neutralization.

When carrying out the third step of the process according to the invention, in general from 0.6 to 2.0 mol, preferably from 0.7 to 0.9 mol, of cyanamide are employed per mole of sodium cyanide.

The dimethyl imidocarbonate neutralized in the second step is metered into the cyanamide solution over a period of from 20 to 120 min, preferably from 30 to 90 min.

In the case of continuous neutralization, the neutralized dimethyl imidocarbonate is metered directly, without buffering, from the neutralization step into the cyanamide, over the period mentioned above.

When the neutralized dimethyl imidocarbonate is metered into the cyanamide, the pH is kept in the neutral range, preferably in the range from pH 6.5 to pH 7.5, particularly preferably in the range from pH 6.8 to pH 7.2, very particularly preferably at pH 7, by addition of further hydrochloric acid.

The process according to the invention is generally carried out under atmospheric pressure. However, if required, the process can also be carried out under elevated or reduced pressure.

Work-up (step four to six of the process according to the invention) is described below.

For carrying out the fourth step of the process according to the invention, it is possible to use, as extractants, all water-immiscible solvents suitable for such reactions. These preferably include aromatic hydrocarbons, such as, for example, benzene, toluene, ethylbenzene, xylene or decalin; halogenated hydrocarbons, such as, for example, chlorobenzene, dichlorobenzene, dichloromethane, chloroform, carbon tetrachloride, dichloroethane or trichloroethane. Particular preference is given to using toluene. The same extractant is also used for step six.

To reduce, in step four, the by-products having oxidizing action, a reducing agent suitable for these purposes, preferably sodium hydrogensulphite, is used.

Work-up is generally carried out such that the DCC suspension obtained after completion of the third step is initially admixed with toluene and then, to reduce by-products having oxidizing action, with sodium hydrogensulphite, and subjected to clarification to remove non-product-containing solid particles. The phases are then separated, the aqueous phase is re-extracted thoroughly and the combined toluene phases are distilled for drying and to remove traces of hydrogen cyanide. The resulting solution of DCC in toluene, having a content of preferably between 10 and 15%, can then be employed directly for subsequent steps, for example a synthesis of an active compound (cf. below).

Owing to the hydrolytic instability of DCC, the work-up described above is carried out continuously to prevent product degradation. Batchwise work-up is possible, but results in loss of yield.

The DCC obtained by the process according to the invention is a known building block for the synthesis of substituted cyanoguanidine compounds which, after further reaction, can be converted into compounds having insecticidal action (cf., for example, EP-A 0 235 725)

### PREPARATION EXAMPLE

Sodium cyanide (95% pure, 169.1 g, 3.28 mol) in 635 ml of water is cooled to −5° C. Aqueous sodium hydroxide solution (45% strength, 320.0 g, 3.6 mol) and 372 g of methanol (11.6 mol) are then added dropwise, and at −5° C. chlorine gas (222.0 g, 3.13 mol) is then introduced above the reaction medium for 10 h. Stirring at −20° C. is continued for a further 16 hours, and the mixture is then, over a period of about 30 min, neutralized with hydrochloric acid (20% strength) until a pH of 7.0 is reached (380 ml, 2.3 mol of hydrochloric acid).

Over a period of 30 min, the suspension is, at −5° C. metered into a solution of cyanamide (110.0 g, 2.62 mol) and water (233 g), and during the addition the pH is maintained at pH 7 using hydrochloric acid (20% strength). The temperature is then allowed to increase to +15° C. over a period of 1 h, and the mixture is stirred at this temperature for another hour. During the entire extra stirring time, the pH is maintained at pH 7 using hydrochloric acid (20% strength). The amount of hydrochloric acid (20% strength solution) consumed during metering in and extra stirring time is about 150 ml.

For work-up, toluene (660 g), Celite 545 (6 g) and sodium hydrogensulphite (80.4 g, 39%) are added to the suspension. The suspension is stirred at room temperature for 30 min, the solid is filtered off, the phases are separated and the aqueous phase is rapidly re-extracted twice with toluene (in each case 430 g). The combined organic extracts are then dried and freed from traces of hydrogen cyanide by distillation.

This gives 1 646 g (13.6% pure, 75% of theory) of dimethyl cyanimidocarbonate (DCC).

1. Process for preparing dimethyl cyanimidocarbonate (DCC, 3,3-dimethoxy-2-azaprop-2-enenitrile) of the formula (I) 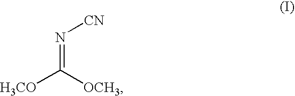
characterized in that
in a first step sodium cyanide is reacted in aqueous sodium hydroxide solution with methanol and chlorine gas and,
in a second step, the resulting dimethyl imidocarbonate (dimethoxymethanimine) of the formula (II) 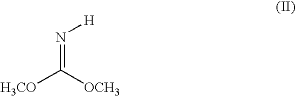
is neutralized and,
in a third step, the resulting neutralized dimethyl imidocarbonate of the formula (II) is introduced into an aqueous solution of cyanamide (aminomethanenitrile), the pH being kept neutral by simultaneous addition of an acid.

2. Process according to claim 1, characterized in that,
in a fourth step, by-products with oxidative action are reduced in the presence of an extractant,
in a fifth step, non-product-containing solid particles are removed by clarification, and,
in a sixth step, DCC of the formula (I) according to claim 1 is concentrated by extraction and distillation.

3. Process according to claim 1 or 2, characterized in that in the first step the chlorine gas is introduced above the reaction mixture.

4. Process according to claim 1 or 2, characterized in that the neutralization and/or work-up is carried out continuously.

5. Process according to claim 1 or 2, characterized in that hydrochloric acid is used for neutralization.

6. Process according to claim 1 or 2, characterized in that the chlorine gas is introduced above the reaction mixture and the neutralization and/or work-up are/is carried out continuously.

7. Process according to claim 2, characterized in that, for the reduction in step 4, sodium hydrogensulphite is used.

8. Process according to claim 3, characterized in that the neutralization and/or work-up is carried out continuously.

9. Process according to claim 3, characterized in that hydrochloric acid is used for neutralization.

10. Process according to claim 4, characterized in that hydrochloric acid is used for neutralization.
